(19) United States
(12) Patent Application Publication
(10) Pub. No.: US 2001/0046957 A1
(43) Pub. Date: Nov. 29, 2001

(54) Method for treating hepatitis C

(76) Inventors: Mary Graves, Welwyn Garden City (GB); Stephen C. Pappas, Clifton, NJ (US); Friederike Zahm, Freiburg (DE)

Correspondence Address: HOFFMANN-LA ROCHE INC., PATENT LAW DEPARTMENT, 340 KINGSLAND STREET, NUTLEY, NJ, 07110

(21) Appl. No.: 09/885,773

(22) Filed: Jun. 20, 2001

Related U.S. Application Data
(63) Continuation of application No. 09/631,579, filed on Aug. 4, 2000.
(60) Provisional application No. 60/187,907, filed on Mar. 8, 2000.

(30) Foreign Application Priority Data
Aug. 13, 1999 (EP) 99115950.0

(51) Int. Cl. A61K 38/00; A01N 37/18

(52) U.S. Cl. 514/2

(57) ABSTRACT

A method of treating a patient infected with a hepatitis C virus to decrease the severity of the viral infection. The method comprises concomitantly administering over a given period of time to the patient a first component and a second component. The first component consists of a pharmaceutical composition containing as an active ingredient a pharmaceutically acceptable salt or prodrug of mycophenolic acid in a therapeutically effective amount to decrease the severity of the viral infection. The second component consists of an injection solution containing as an active ingredient interferon-α or pegylated interferon-α in a therapeutically effective amount to decrease the severity of the viral infection. The components are concomitantly administered over a period of time at least sufficient to reduce the amount of HCV-RNA present in the peripheral blood of said patient to less than 100 copies/ml at 24 weeks after the end of treatment.

### Cross Reference to Related Application

This is a continuation of copending patent application No. 09/631,579, filed Aug. 4, 2000.

### Field of the Invention

The invention relates to the field of treating liver diseases, in particular hepatitis C infections, by administering (i) interferon-α or pegylated interferon-α and (ii) a pharmaceutically acceptable salt or a prodrug of mycophenolic acid.

### BACKGROUND OF THE INVENTION Background of the Invention

Hepatitis C virus (HCV) is a liver damaging infection that can lead to cirrhosis, liver failure or liver cancer. It is currently estimated that there are 170 -200 million people infected with this virus worldwide.

Interferons (IFNs) is a family of naturally occurring small proteins with characteristic biological effects such as antiviral, immunoregulatory and antitumoral activities. They are produced and secreted by most animal nucleated cells in response to several diseases, in particular viral infections.

Four distinct classes of human IFNs are known to exist (Pestka et al); (Emanuel and Pestka). In the family of IFNs, the IFN-α family represents the predominant class of IFNs produced by stimulated peripheral blood leukocytes, and lymphoblastoid and myeloblastoid cell lines.

IFN-α has emerged as an important regulator of growth and differentiation affecting cellular communication and immunology control. IFN-α is frequently used in the treatment of chronic hepatitis such as chronic HCV infection (CHC).

In fact, IFNs remain the only approved monotherapy for chronic HCV infection (CHC). The goal of treatment is to achieve a sustained virological response (that is non-detectable (<100 copies/ml) of HCV-RNA in peripheral blood) at 6 months after the end of treatment. However, the effectiveness of IFN in the treatment of CHC is unsatisfactory. IFN-α monotherapy results in a sustained response in only 5-20% of general CHC populations. (Fried M). Further, IFNs typically cause flu-like symptoms at the onset of treatment.

It is believed that IFNs do not attain maximum clinical potency because, inter alia, IFNs are rapidly cleared from the systemic circulation. It has been found that for INFα conjugation with polyethylene glycol (PEG) reduces clearance. Furthermore, it has recently been shown that a PEG IFN-α2A conjugate caused sustained reduction of the virus to undetectable levels in 36% of non cirrhotic with CHC in a phase II study. (Shiffman M). A subsequent phase III study in CHC patients with cirrhosis demonstrated a sustained virological response of 29%. (Heathcote J).

Ribavirin (1-β-D-ribofuranosyl-1 H-1 ,2,-4-triazole-3-carboxamide), an inhibitor of inosine 5′-monophosphate dehydrogenase (IMPDH), enhances the efficacy of IFNα in the treatment of CHC. Sustained virological response rates improve approximately 2-fold over IFN-α monotherapy. (Lai MY); (Reichard O). This regimen showed efficacy in achieving sustained loss of detectable virus in up to 30 - 40% of treated patients. (McHutchison JG). However, most patients still either did not respond or tolerated treatment poorly. When combined with a pegylated form of IFN-α2A, an end of treatment virological response was achieved in 70% of patients in a small pilot study of safety. (Sulkowski M.). A number of patients still had significant side effects, primarily related to ribaviran. Ribavirin causes significant hemolysis in 10-20% of patients treated at currently recommended doses, and the drug is both teratogenic and embryotoxic.

Mycophenolic acid (MPA) is an active ingredient that inhibits the proliferation of B and T lymphocytes through noncompetitive and reversible inhibition of inosine monophosphate dehydrogenase (IMP-DH), a key enzyme in the novo synthetic pathway of guanidine nucelotides. In particular, MPA is a potent, selective, non-competitive inhibitor of the type 2 isoform of IMPDH expressed in activated T and B lymphocytes.

However, the bioavailability of MPA is limited, thus preventing it from attaining its maximum clinical potency. Prodrugs and salts of MPA have been shown to possess clinically useful properties. One such prodrug, the morpholinoethyl ester of MPA, also known as mycophenolate mofetil (MMF) is commercially available as an immunosuppressant for the treatment of refractory rejection, particularly, in liver transplant recipients. It has been used in monotherapy or in combination with cyclosporin and corticosteroids.

MMF has been shown to have antiviral activity (Neyts J) and there is an anecdotal report that serum HCV RNA levels decrease in some patients with CHC virus infection following liver transplant after initiation of MMF therapy for treatment of rejection. (Platz KP). However, no clinical studies have been published regarding MMF in CHC patients.

IFN-α or PEG IFN-α in association with MPA or one of its prodrugs or salts (such as MMF) may be of importance in the treatment of liver diseases and, in particular, CHC.

### SUMMARY OF THE INVENTION

The present invention provides the use of INF-α or PEG INF-α in association with MPA or one of its prodrugs or salts (for example, MMF) for the manufacture of medicaments for treating liver disease. The present invention has also for an object medicaments containing INF-α or PEG INF-α and MPA or one of its prodrugs or salts (for example, MMF) as a combined preparation for simultaneous, part-simultaneous, separate or sequential use in therapy of liver diseases. The present invention also concerns a method for treating a liver disease patient comprising administering to the patient INF-α or PEG INF-α in association with MPA or one of its prodrugs or salts (for example, MMF).

A dosage of IFN-α for practicing the combination therapy of this invention is from 3 to 6 million international units (IU) administered three times weekly. A preferred dosage for practicing the combination therapy of this invention is 3 million IU administered three times daily. A dosage of PEG INF-α for practicing the combination therapy of this invention is from 40 to 270 μg administered once per week. A preferred dosage is 180 μg administered once per week. A dosage for MPA or one of its prodrugs or salts (for example, MMF) for practicing the invention is from 250 to 2000 mg per day, preferably 500-1000 mg. This daily dosage may be administered in divided doses twice to four times per day.

For purposes of conversion, 1 mg of INF-α equals 2.7×10⁸ IU. Thus, 3 million IU of INF-α equals 11.1 μg of IFN-α.

In particular, the present invention relates to a method of treating a patient infected with HCV to decrease the severity of the viral infection. The method comprises concomitantly administering to the patient a first component consisting of a pharmaceutical composition containing as an active ingredient a prodrug or pharmaceutically acceptable salt of MPA in a therapeutically effective amount to decrease the severity of the viral infection and a second component consisting of an injection solution containing as an active ingredient INF-α or pegylated IFN-α conjugate in a therapeutically effective amount to decrease the severity of the viral infection. The components are administered over a period of time at least sufficient to reduce the amount of HCV-RNA present in the peripheral blood of the patient to less than 100 copies/ml after the period of time.

In a particular aspect, the present invention relates to a method of treating a patient infected with a hepatitis C virus, comprising concomitantly administering to the patient:

(i) a first component consisting of pharmaceutical composition containing as an active ingredient a pharmaceutically acceptable salt of the formula:

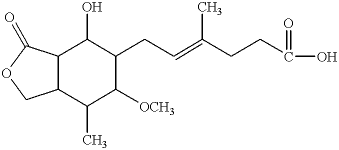

or a compound of the formula

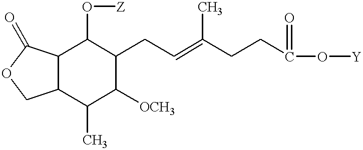

wherein Y is

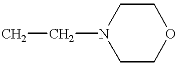

and Z is hydrogen or —(CO)R and R is lower alkyl or aryl,

wherein the active ingredient of the first component is administered daily in an amount of from about 3 mg/kg to about 40 mg/kg, and

(ii) a second component consisting of an injection solution containing as an active ingredient interferon-α or pegylated interferon-α conjugate, wherein the active ingredient of the second component is administered weekly in amount of from about 0.5 μg/kg to about 3.6 μg/kg,

said components being concomitantly administered over a period of time from about 24 weeks to about 72 weeks.

It was unexpectedly found that administration of the two components in accordance with the present invention results in a reduction of the amount of HCV-RNA present in the peripheral blood to less than 100 copies/ml for example at 24 weeks after the end of administration.

In another aspect, the invention relates to a kit. The kit comprises a first compenent and a second component. The first component contains one or more oral unit dosage forms of an active ingredient, each unit containing the active ingredient in a amount of from about 250 mg to about 2000 mg, wherein the active ingredient is of pharmaceutically acceptable salt or prodrug of MPA. The second component contains a vial or series of vials each vial containing a single injectable solution dose or multiple injectable solution doses, each dose containing as an active ingredient about 40 μg to about 270 μg of interferon-α or pegylated interferon-α.

In a particular aspect, the invention relates to a kit comprising a first component and a second component. The first component contains one or more oral unit dosage forms of an active ingredient, each unit containing about 250 mg to about 2000 mg of the active ingredient, wherein the active ingredient is a compound of the formula,

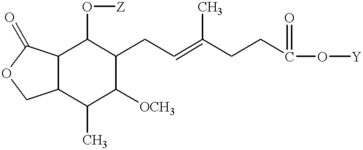

wherein Y is

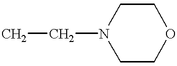

and Z is hydrogen. The second component contains a vial or series of vials each vial containing a single injectable solution dose or multiple injectable solution doses, each dose containing as an active ingredient about 40 μg to about 270 μg of a pegylated interferon-α conjugate.

### DETAILDE DESCRIPTION OF THE INVENTION

To practice the invention, INF-α or PEG INF-α in association with MPA or one of its prodrugs or salts (for example, MMF) are administered to patients suffering liver diseases. Particularly, the association of INF-α or PEG INF-α in association with MPA or one of its prodrugs or salts (for example, MMF) as described in the present invention is efficacious for treating viral infections and, especially CHC.

MPA is a known compound of formula:

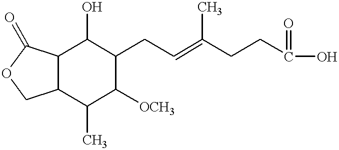

The first component of the present invention consists of a pharmaceutical composition containing as an active ingredient a prodrug or pharmaceutically acceptable salt of MPA in a therapeutically effective amount to decrease the severity of the viral infection.

The term “pharmaceutically acceptable salt of MPA” as used herein is any conventional salt or base addition salt that retains the biological effectiveness and properties of MPA and which is formed from a suitable non-toxic organic or inorganic acid or organic or inorganic base. Preferred are cationic salts, for example, of alkali metals, especially sodium salts. Sodium mycophenolate salts are known, for example in WO 97/38689.

The term “prodrug of MPA” as used herein refers to a compound that is converted under physiological conditions or by solvolysis to MPA. A prodrug of MPA may be inactive when administered to a subject but is converted in vivo to MPA.

Preferred as a prodrug of MPA is a compound of the formula:

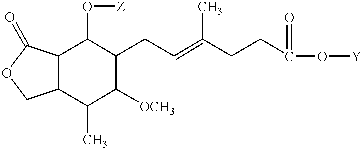

wherein Y is

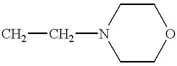

and Z is hydrogen or —(CO)R and R is lower alkyl or aryl.

These compounds are known from U.S. Pat. No. 4,753,935, incorporated herein by reference. Most preferred is the compound MMF (Z is hydrogen).

The second component consisting of an injection solution containing as an active ingredient INF-α or pegylated INF-α conjugate in a therapeutically effective amount to decrease the severity of the viral infection.

The term “INF-α” as used herein includes INF-αs derived from any natural material (for example, leokocytes, fibroblasts, lymphocytes) or material derived therefrom (for example, cell lines), or those prepared with recombinant DNA technology. Any protein having activity of INFα, such as muteins or otherwise modified proteins, is encompassed. Obtaining and isolating IFNα from natural or recombinant sources is well known (Pestka I) (Goeddel I) (Goeddel II) (EP 32134, EP 43980, EP 211148). There are many types of INFα such as IFN-α1, IFN-α2 and further their subtypes including but not limited to IFN-α2a, IFN-α2b, IFN-α2c, and IFN-α2II (also designated IFN-αII or w-IFN). In the present invention, the use of IFN-α2a is preferred. The manufacture of IFN-α2a is known (Pestka II) (EP 43980, EP 211148).

The term “pegylated INF-α conjugate” as used herein includes any interferon-α that is conjugated to a substituted or unsubstituted polyalkylene glycol group, for example, a polyethylene glycol (PEG) group. The molecular weight of the polymer, which is preferably PEG, may range from 300 to 30,000 Dalton.

A PEG group is conjugated to the INF-α via a covalent linkage of INF-α to PEG which has been activated by replacement of the PEG hydroxyl with a linking group, forming a reagent which is an N-hydroxy succinamide ester derivative of PEG. The reagent may be obtained by conventional means (Monfardini). Linkage is via an amide or ester bond. Preferably, linkage via an amide bond. Linkers known in the art include those disclosed in EP′ Publication Nos. 0510356 and 593868 and EP-A 9/108261.5.

Most preferably, the reagents attach to primary amino groups on for example lysine or to the N-terminus of the IFN-α. The reagents can also attach to a hydroxyl on for example serine. One or more, preferably one to three, PEGs may be conjugated to the IFN-α.

A most preferred reagent is of the formula

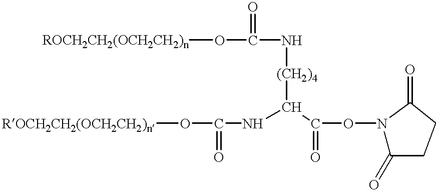

in which a total of 2 monomethoxy PEG (m-PEG) chains is linked to lysine, one at the α and ε amino groups via carbamate (urethane) bonds and the lysine carboxyl group is activated to a succinimidyl ester. This reagent may be obtained by conventional means, according to known procedures (Monfardini) applicable to a reagent with R being lower alkyl and having a desired n. This reagent may be obtained from Shearwater Polymers, Inc. (Huntsville, Ala.). The preferred average molecular weight of the PEG is about 20,000 daltons, providing a total PEG mass of about 40,000 daltons in PEG2-NHS (other molecular weights may be obtained by varying n for the PEG-alcohol starting materials for the reagent of the above formula, by conventional means.

A preferred pegylated-INF-α conjugate has the formula:

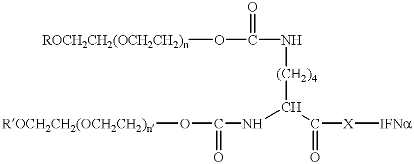

wherein R and R′ are independently lower alkyl; X is NH or O; n and n′ are integers having a sum of from 600 to 1500; and the average molecular weight of the polyethylene glycol in said conjugate is from about 26,000 daltons to about 66,000 daltons.

Most preferred is the pegylated interferon-α is of the formula

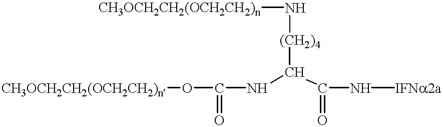

wherein n and n′ are independently 420 or 520. This pegylated INF-α conjugate is known, for example in U.S. patent application Ser. No. 09/255,948, filed Feb. 23, 1999, incorporated herein by reference.

In accordance with the present invention, administration of the two components synergistically enhances the treatment of hepatitis C provided by administering each component separately. The synergistic effect results in a sustained response. By “sustained response” is meant that the amount of HCV in peripheral blood set forth as copies of HCV-RNA in peripheral blood is less than 100 copies/ml, for example when measured at 24 weeks after the end of administration of the two components.

The amount of HCV in peripheral blood set forth as copies of HCV-RNA/ml is determined by known methods. In vitro diagnostic kits for determining the amount are commercially available, such as the Amplicor® HCV Monitor Test (a quantitative test sensitive to 1000 copies/ml), the Amplicor® Hepatitis C Virus (HCV) Test (a qualitative test sensitive to 100 copies/ml), the Cobas Amplicor™ Hepatitis C Virus Test (an automated qualitative test sensitive to 100 copies/ml), and the Cobas Amplicor™ HCV Monitor Test (an automated quantitative test sensitive to 1000 copies/ml) (each Test may be obtained from Roche Diagnostic Systems, Inc., Branchburg, N.J.). A preferred method for determining the amount is set forth in the Example.

The injection solution of INF-α or pegylated INF-α is administered to the patient parenterally, preferably by subcutaneous (sc) or intramuscular (im) injection. Preferably, the pharmaceutically acceptable salt to or prodrug of MPA is administered to the patient in an oral unit dosage form, more preferably in capsule, pill, sachet or tablet form in association with the parenteral administration of INF-α or pegylated IFN-α.

Of course, other types of administration of both medicaments, as they become available are contemplated, such as by nasal spray, transdermally, by suppository, by sustained release dosage form, etc. Any form of administration will work so long as the proper dosages are delivered without destroying the active ingredients.

The first component and the second component of the present invention are administered in any amount and for any duration that is effective to decrease the severity of the hepatitis C infections.

Generally, it is preferred that administration of the first component and the second component occur concomitantly for a period of from about 24 to about 72 weeks, preferably from about 24 to about 48 weeks, and most preferably for about 48 weeks.

Generally, the dosage for the active ingredient of the injection solution (INF-α or pegylated IFN-α) is about 0.5 μg/kg to about 3.6 μg/kg of body weight, administered about 1 time per week. Generally, the dosage for the pharmaceutically acceptable salt or prodrug of MPA is about 3 mg/kg to about 40 mg/kg, preferably about 5 mg/kg to about 36 mg/kg, and most preferably about 12 mg/kg to about 25 mg/kg, administered daily. The dosage levels may be modified by the physician to be lower or higher than that stated herein depending on the needs of the patient, and the reaction of the patient to the treatment.

The dosages may be administered according to any dosage schedule determined by the physician in accordance with the requirements of the patient. For example, the dosages of each of the two components may be administered in single or in divided doses.

The therapeutically effective amount of IFN-α or PEG IFN-α and the therapeutically effective amount of MPA or one of its prodrugs or salts (for example, MMF) may be administered simultaneous, part-simultaneous, separate or sequential. For example, the therapeutically effective of MPA or one of its prodrugs or salts (for example, MMF) may be administered to the patient in association with the therapeutically effective amount of INF-α or PEG IFN-α, that is the IFN-α or PEG IFN-α dose may be administered during the same or different periods of time that the patient receives doses of MPA or one of its prodrugs or salts (for example, MMF).

In accordance with the present invention, a kit, useful for treating hepatitis C, is provided. The kit comprises a first component and a second component. The first component contains one or more oral unit dosage forms of an active ingredient, each unit containing about 250 mg to about 2000 mg (preferably 500-1000 mg) of the active ingredient, wherein the active ingredient is a pharmaceutically acceptable salt or prodrug of MPA. The second component contains a vial or series of vials each vial containing a single injectable solution dose or multiple injectable solution doses, each dose containing as an active ingredient about 40 μg to about 270 μg (preferably 180 μg ) of interferon-α or a pegylated interferon-α conjugate.

Preferably, the first component contains a sufficient number of units so that a patient can administer about 2 grams per day of the active ingredient for a period of about 1 to about 4 weeks and the second component contains a sufficient number of doses so that a patient can administer about 180 μg per week of interferon-α or a pegylated interferon-α conjugate for a period of about 1 to about 4 weeks.

Preferably, the active ingredient of the first component is a prodrug of mycophenolate mofetil, having the formula

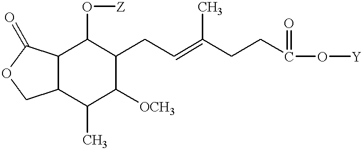

wherein Y is

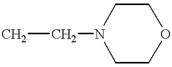

and Z is hydrogen or —(CO)R and R is lower alkyl or aryl.

Most preferably, the active ingredient of the first component is a compound of the formula

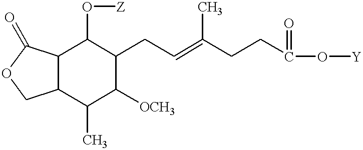

wherein Y is

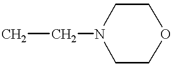

and Z is hydrogen.

Preferably, the active ingredient of each injectable solution dose is a pegylated interferon-α2a conjugate, more preferably a pegylated interferon-α2a conjugate of the formula

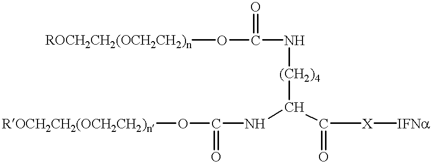

wherein R and R′ are independently lower alkyl; X is NH or O; n and n′ are integers having a sum of from 600 to 1500; and the average molecular weight of the polyethylene glycol in said conjugate is from about 26,000 daltons to about 66,000 daltons.

Most preferably, the active ingredient of each injectable solution dose is a pegylated interferon-α2a conjugate of the formula

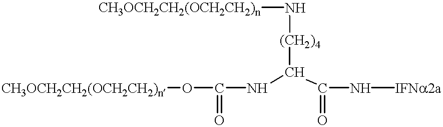

wherein n and n′ are independently 420 or 520.

The present invention may be exemplified by controlled clinical trials as shown in the Example below, which illustrates the invention without limitation.

### EXAMPLE

### Patients

60 patients are treated. All patients have serological evidence of HCV, have serum HCV-RNA quantifiable at ≧2000 copies/ml, have elevated serum alanine aminotransferase (ALT) activity documented during the 35 day period preceding the initiation of test drug dosing, and have chronic liver disease consistent with chronic HCV infection on a biopsy obtained within the 24 months preceding the initiation of test drug dosing.

Patients may not have other forms of liver disease (including cirrhosis), anemia, hepatocellular carcinoma, pre-existing severe depression or other psychiatric disease, cardiac disease, renal disease, seizure disorders, or sever retinopathy.

Drug Formulations
Tablet Formulation
Formulation 1

| Ingredient | mg/Tablet |
|---|---|
| MMF | 500.00 |
| Microcrystalline cellulose | 244.00 |
| Croscarmellose sodium | 32.50 |
| Povidone K90 | 24.40 |
| Magnesium Stearate | 12.20 |
| TOTAL | 813.10 |
| Purified Water | q.s. |

Formulation 2

| Ingredient | Unit Formula per mL | |
|---|---|---|
| Peginterferon alfa-2a bulk solution¹ | 360 | g |
| Sodium Chloride² | 8.0 | g |
| Benzyl Alcohol | 10.0 | mg |
| Sodium Acetate trihydrate | 2.617 | mg |
| Acetic Acid (glacial)² | 0.0462 | mg |
| Polysorbate 80 | 0.05 | mg |

| | |
|---|---|
| Sodium Acetate trihydrate 10%³ | q.s. to pH 6.0 0.2 |
| Acetic Acid 10%³ | q.s. to pH 6.0 0.2 |
| Water for Injection⁴ | q.s. to 1.0 mL (1.004 g⁴) |

¹Bulk PEG-IFN is supplied as an aqueous solution in 20 mM sodium acetate, buffered at pH 6, and containing 50 mM sodium chloride; the required amount is calculated using the actual protein content and the actual density of the bulk drug substance. The theoretical protein content of PEG-IFN is 1.3 mg/mL and the theoretical density is 1.002 g/mL.
²Correction must be made for the amount of sodium chloride and sodium acetate and glacial acetic acid already included in the bulk drug substance.
³An equivalent amount of a different concentration can be used.
⁴Density (20C) of the drug product solution is 1.004 g/mL.

### Treatment

Each patient is orally administered formulation 1, two times per day for 48 weeks. Concominantly, each patient is administered formulation 2 as a subcutaneous injection, once weekly for 48 weeks.

### Primary Efficacy Parameter

The primary efficacy parameter is a sustained virological response rate (that is non-detectable (<100 copies/ml) HCV-RNA at the conclusion of a 24 week treatment-free follow up period).

To detect HCV-RNA, HCV-RNA is first isolated from serum or plasma by lysis of virus particles with a chaotropic agent followed by precipitation of the RNA with alcohol. A second target sequence (Standard) is introduced with the lysis reagent. Preferably, the Standard is a non-infectous, 351 nucleotide in vitro transcribed RNA molecule with primer binding regions identical to those of the HCV target sequence. Preferably, the Standard contains KY78 and KY80 primer binding regions and generates a product of the same length (244 bases) and base composition as the HCV target RNA. The probe binding region of the Standard amplicon is amplified to differentiate Standard amplicon from HCV target amplicon. The Standard is carried through the specimen preparation, reverse transcription, amplification and detection steps. The Standard compensates for effects of inhibition and controls for the amplification process to permit the accurate quantitation of HCV-RNA.

Selection of the target RNA sequence for HCV depends on identification of regions within the HCV genome that show maximum conservation among the various HCV genotypes. The 5′-untranslated region of the HCV genome has been shown to have maximum conservation of RNA sequence among known HCV genotypes. Preferably used are primers KY78 and KY80 to define a sequence of 244 nucleotides within the highly conserved 5′-untranslated region of the HCV genome. (Young, K.) The capture probe sequence and the primer sequences are located in the most conserved domains within the 5′-untranslated region. (Bukh, J.)

Reverse transcription and amplification reactions are performed with the thermostable recombinant enzyme Thermus thermophilus DNA Polymerase (rTth pol). In the presence of maganese and under the appropriate buffer conditions, rTth pol has both reverse transcriptase and DNA polymerase activity. This allows both reverse transcription and PCR amplification to occur in the same reaction mixture.

Processed specimens are added to the amplification mixture in reaction tubes in which both reverse transcription and PCR amplification occur. The downstream or antisense primer (KY78) is biotinylated at the 5′ end; the upstream or sense primer (KY80) is not biotinylated. The reaction mixture is heated to allow the downstream primer to anneal specifically to the HCV target RNA and to the HCV Standard RNA. In the presence of excess deoxynucleoside triphosphates (dNTPs), including deoxyadenosine, deoxyguanosine, deoxycytidine and deoxyuridine (in place of thymidine) triphosphates, rTth pol extends the annealed primer forming a DNA strand (cDNA) complementary to the RNA target.

Following reverse transcription of the HCV target RNA and the HCV Standard RNA, the reaction mixture is heated to denature the RNA:cDNA hybrid and expose the primer target sequences. As the mixture cools, the upstream primer (KY80) anneals specifically to the cDNA strand, rTth pol extends the primer, and a second DNA strand is synthesized. This completes the first cycle of PCR, yielding a double-stranded DNA copy of the target region of the HCV and Standard RNA. The reaction mixture is heated again to separate the resulting double-stranded DNA and expose the primer target sequences. As the mixture cools, the primers KY78 and KY80 anneal to the target DNA. RTth pol, in the presence of excess dNTPs, extends the annealed primers along the target templates to produce a 244 base pair double-stranded DNA molecule termed an amplicon. This process is repeated for a designated number of cycles, each cycle effectively doubling the amount of amplicon DNA. Amplification occurs only in the region of the HCV genome between the primers; the entire HCV genome is not amplified.

Selective amplification of target nucleic acid from the clinical specimen is achieved by the use of uracil-N-glycosylase, UNG and deoxyuridine triphosphate (dUTP). Uracil-N-glycosylase, UNG recognizes and catalyzes the destruction of DNA strands containing deoxyuridine, but not DNA containing thymidine. Deoxyuridine is not present in naturally occurring DNA, but is always present in amplicon due to the use of deoxyuridine triphospate in place of thymidine triphosphate as one of the dNTPs in the Master Mix reagent; therefore, only amplicon contain deoxyuridine. Deoxyuridine renders contaminating amplicon susceptible to destruction by uracil-N-glycosylase, UNG prior to amplification of the target DNA. Uracil-N-glycosylase, UNG, which is included in the Master Mix reagent, catalyzes the cleavage of deoxyuridine containing DNA at the deoxyuridine residues by opening the deoxyribose chain at the C-1 position. When heated in the first thermal cycling step at the alkaline pH of Master Mix, the amplicon DNA chain breaks at the position of the deoxyuridine, thereby rendering the DNA non-amplifiable. Uracil-N-glycosylase, UNG is inactive at temperatures above 55° C., i.e., throughout the thermal cycling steps, and therefore does not destroy target amplicon. Following amplification, any residual enzyme is denatured by the addition of a denaturation solution, thereby preventing the degradation of any target amplicon. Uracil-N-glycosylase, UNG has been demonstrated to inactivate at least 10³ copies of deoxyuridine-containing HCV amplicon per PCR.

Following PCR amplification, the HCV amplicon and the Standard amplicon are chemically denatured to form single-stranded DNA by the addition of denaturation solution. Aliquots of denatured amplicon are added to separate wells of a microwell place (MWP) coated with HCV-specific (for example, KY150) and Standard-specific (for example, SK535) oligonucleotide probes. HCV and Standard amplicon are bound to HCV and Standard wells, respectively, by hybridization to the MWP-bound oligonucleotide probes. To achieve quantitative results over a large dynamic range, serial dilutions of the denatured amplicon are analyzed in the MWP.

Following the hybridization reaction, the MWP is washed to remove any unbound material and Avidin-horseradish peroxidase conjugate is added to each well of the MWP. The Avidin-horseradish peroxidase conjugate binds to the biotin-labeled amplicon captured by the target-specific oligonucleotide probes (HCV or Standard) bound to the MWP. The MWP is washed again to remove unbound conjugate and a substrate solution containing hydrogen peroxide and 3,3′,5,5′-tetramethylbenzidine (TMB) is added to the wells. In the presence of hydrogen peroxide, the bound horseradish peroxidase catalyzes the oxidation of TMB to form a colored complex. The reaction is stopped by the addition of a weak acid and the optical density is measured at 450 nm using an automated microwell plate reader.

Within the linear range of the assay, the optical density (OD) in each well of the MWP is proportional to the amount of HCV amplicon or Standard amplicon in the well. The calculated total OD is proportional to the amount of HCV RNA or Standard RNA, respectively, present in each reverse transcription/PCR amplification reaction. The amount of HCV RNA in each specimen is calculated from the ratio of the total HCV OD to the total Standard OD and the input number of Standard RNA molecules using the following equation:

$$\frac{Total HCV OD}{Total QS OD} \times Input HCV QS copies / PCR \times 200 = HCV RNA copies / ml$$

Where:

Total HCV OD=calculated total OD for HCV amplicon

Total QS OD=calculated total OD for QS amplicon

Input HCV QS copies/PCR=the number of copies of QS in each reaction;

200=factor to convert copies/PCR to copies/mL

### Results

It was unexpectedly found that in more than 20% of the patients treated, administration of the two formulations in accordance with the above example resulted in a reduction of the amount of HCV-RNA present in the peripheral blood to less than 100 copies/ml at 24 weeks after the end of treatment.

### References

1. Pestka et al. (1987) Ann. Rev. Biochem, 56, 727-777.

2. Emanuel and Pestka (1993), J. Biol. Chem., 268,12565-12569.

3. Fried M and Hoofnagle J., “Therapy of hepatitis C”, Semin. Liver Dis., 1995, 15(1), 82-91

4. Shiffman M, Pockros P J, Reddy R K et al., “A controlled, randomized, multicenter, ascending dose phase II trial of pegylated interferon alfa-2a (PEG) vs. standard interferon alfa-2a (IFN) for treatment of chronic hepatitis C”, Gastroneterology 1999; 116 (pt 2): 1275. Abstract LO418.

5. Heathcote J, Shiffman M L, Cooksley G, et al., “Multinational evaluation of the efficacy and safety of once weekly PEG interferon alpha-2a (PEG-IFN) in patients with chronic hepatitis C (CHC) with compensated cirrhosis.” Hepatology 1999;30 (suppl):316A.

6. Lai M Y, Kao J H, Yang P M, et al., “Long term efficacy of ribaviran plus interferon alfa in the treatment of chronic hepatitis C”, Gastroenterology 1996, 111, 1307-1312.

7. Reichard O, Norkrans G, Fryden A, et al., “Randomized, double-blind, placebo controlled trial of interferon α-2b with and without ribavirin for chronic hepatitis C”, Lancet 1998, 351, 83-87).

8. McHutchinson J G, Gordon S, Schiff E R, et al., “Interferon alfa-2a monotherapy versus interferon alfa-2b plus ribaviran as initial treatment for chronic hepatitis C: Results of a U.S. multicenter randomized controlled study”, New Engl. J. Med., 1998, 339, 1485-92.

9. Sulkowski M, Reindollar R., “Combination therapy with peginterferon alfa-2a (PEG-IFN) and ribavirin in the treatment of patients with chronic hepatitis C (CHC): a phase II openlabel study.” Hepatology 1999;30(suppl): 197A.

10. Neyts J, Meerbach A, McKenna P, DeClercq E, “Use of yellow fever virus vaccine strain 17D for the study of strategies for the treatment of yellow fever virus infections”, Antiviral Res, 1996, 30:125-32).

11. Platz K P, Mueller A R, Willimski B, Mansoorian, Berg T, Neuhaus R, Hopf U, Lobeck H, “Indications for mycophenolate mofetil therapy in hepatitis C patients undergoing liver transplantation”, Transpl. Proc. 1998, 30:1468-9).

12. S. Pestka (I) The human interferons from protein purification and sequence to cloning and expression in bacteria: before, between, and beyond. Arch. Biochem. Biocphys. 221:1 (1983).

13. Goeddel (I) (1980) Nature 284, 316-320.

14. Goeddel (II) (1981), Nature 290, 20-26.

15. S. Pestka (II), The purification and manufacture of human interferons. Sci. Am. 249:36 (1983).

16. C. S. Monfardini, O. Schiavon, P. Caliceti, M. Morpurgo, J. M. Harris and F. M. Veronese, A branched monomethoxypoly(ethylene glycol) for protein modification. Bioconjugate Chem. 6:62 (1995).

17. Young, K., Resnick, and Myers, T., 1992. Detection of Hepatitis C Virus RNA by combined reverse transcriptase-polymerase chain reaction assay, Journal of Clinical Microbiology 31:882-886).

18. Bukh, J., Purcell, R. H., and Miller R. H., 1992. Sequence analysis of the 5′ noncoding region of hepatitis C virus, Proceedings of the National Academy of Sciences, USA 89:4942-4946).

1. A method for treating a patient infected with a liver disease to decrease the severity of the disease, comprising administering to said patient a therapeutically effective amount of interferon-α or pegylated interferon-α in association with a therapeutically effective amount of a pharmaceutically acceptable salt or prodrug of mycophenolic acid.

2. The method of claim 1, wherein the liver disease is a viral infection.

3. The method of claim 2, wherein the viral infection is chronic hepatitis C.

4. The method of claim 3, wherein the interferon-α or pegylated interferon-α is interferon-α2A or pegylated interferon-α2A.

5. The method of claim 4, wherein a part of the therapeutically effective amount of the pharmaceutically acceptable salt or prodrug of mycophenolic acid is first administered followed by a combination of the remainder of the therapeutically effective amount of the pharmaceutically acceptable salt or prodrug of mycophenolic acid in association with the therapeutically effective amount of interferon-α2A or pegylated interferon-60 2A.

6. The method of claim 5, wherein the pharmaceutically acceptable salt or prodrug of mycophenolic acid is administered orally.

7. The method of claim 6, wherein the interferon-α2A or pegylated interferon-α 2A is administered parenterally.

8. The method of claim 7, wherein the amount of a pharmaceutically acceptable salt or prodrug of mycophenolic acid is from about 3 mg/kg/day to about 40 mg/kg/day.

9. The method of claim 8, wherein the amount of interferon-α or pegylated interferon-α is from about 0.5 μg/kg/week to about 3.6 μg /kg/week.

10. A method of claim 3, comprising concomitantly administering over a given period of time to the patient a first component consisting of pharmaceutical composition containing as an active ingredient a pharmaceutically acceptable salt or prodrug of mycophenolic acid in a therapeutically effective amount to decrease the severity of the viral infection and a second component consisting of an injection solution containing as an active ingredient interferon-α or pegylated interferon-α in a therapeutically effective amount to decrease the severity of the viral infection, said components being concomitantly administered over a period of time at least sufficient to reduce the amount of HCV-RNA present in the peripheral blood of said patient to less than 100 copies/ml after said period of time.

11. The method of claim 10, wherein the pharmaceutical composition of the first component is an oral unit dosage form.

12. The method of claim 11, wherein second component consists of an injection solution containing as an active ingredient pegylated interferon-α.

13. The method of claim 12, wherein the pegylated interferon-α is a conjugate of the formula 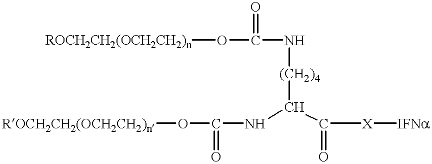
wherein R and R′ are independently lower alkyl; X is NH or O; n and n′ are integers having a sum of from 600 to 1500; and the average molecular weight of the polyethylene glycol in said conjugate is from about 26,000 daltons to about 66,000 daltons.

14. The method of claim 13, wherein the pegylated interferon-α is of the formula 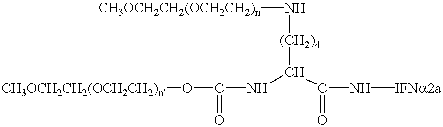
wherein n and n′ are independently 420 or 520.

15. The method of claim 14, wherein the active ingredient of the first component is a pharmaceutically acceptable salt of the formula: 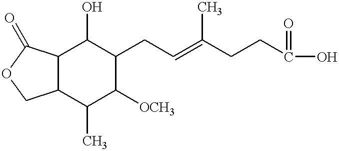
or a compound of the formula 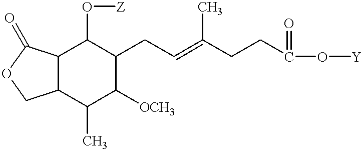
wherein Y is 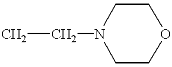
and Z is hydrogen or —(CO)R and R is lower alkyl or aryl.

16. The method of claim 15, wherein the active ingredient of the first component is a compound of the formula 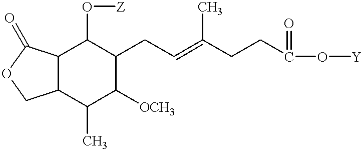
wherein Y is 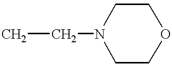
and Z is hydrogen.

17. A method of treating a patient infected with a hepatitis C virus, comprising concomitantly administering to the patient:
(i) a first component consisting of pharmaceutical composition containing as an active ingredient a pharmaceutically acceptable salt of the formula: 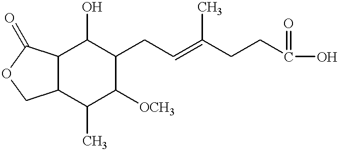
or a compound of the formula 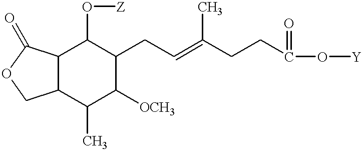
wherein Y is 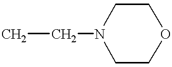
and Z is hydrogen or —(CO)R and R is lower alkyl or aryl,
wherein the active ingredient of the first component is administered in an amount of from about 3 mg/kg to about 40 mg/kg per day, and
(ii) a second component consisting of an injection solution containing as an active ingredient interferon-α or pegylated interferon-α wherein the active ingredient of the second component is administered in amount of from about 0.5 μg/kg to about 3.6 μg/kg per week,
said components being concominantly administered over a period of time from about 24 weeks to about 72 weeks.

18. The method of claim 17, wherein the active ingredient of the second component is a pegylated interferon-α conjugate of the formula 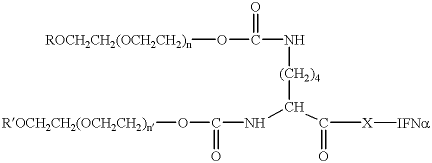
wherein R and R′ are independently lower alkyl; X is NH or O; n and n′ are integers having a sum of from 600 to 1500; and the average molecular weight of the polyethylene glycol in said conjugate is from about 26,000 daltons to about 66,000 daltons.

19. The method of claim 18, wherein the active ingredient of the pharmaceutical composition of the first component is administered in an amount of from about 12 mg/kg to about 25 mg/kg per day.

20. The method of claim 18, wherein the active ingredient of the pharmaceutical composition of the first component is administered in an amount of about 500 mg per day.

21. The method of claim 18, wherein the active ingredient of the pharmaceutical composition of the first component is administered in an amount of about 250 mg per day.

22. The method of claim 18, wherein the components are concominantly administered over a period of time from about 24 weeks to about 48 weeks.

23. The method of claim 18, wherein the components are concominantly administered over a period of time for about 48 weeks.

24. The method of claim 23, wherein the active ingredient of the first component is a compound of the formula 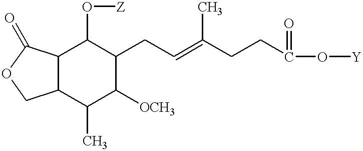
wherein Y is 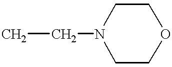
and Z is hydrogen.

25. The method of claim 24, wherein the active ingredient of the second component is a pegylated interferon-α conjugate of the formula 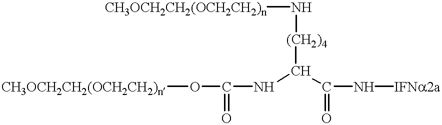
wherein n and n′ are independently 420 or 520.

26. A kit comprising:
(a) a first component containing one or more oral unit dosage forms of an active ingredient, each unit containing about 250 mg to about 2000 mg of the active ingredient, wherein the active ingredient is a pharmaceutically acceptable salt or prodrug of mycophenolic acid, and
(b) a second component containing a vial or series of vials, each vial containing a single injectable solution dose or multiple injectable solution doses, each dose containing as an active ingredient about 40 μg to about 270 μg of interferon-α or pegylated interferon-α.

27. The kit of claim 26, wherein the first component contains a sufficient a sufficient number of units so that a patient can administer about 2 grams per day of the pharmaceutically acceptable salt or prodrug of mycophenolic acid for a period of about one to about four weeks and the second component contains a sufficient number of doses so that a patient can administer about 180 μg per week of interferon-α or pegylated interferon-α for a period of about one to about four weeks.

28. The kit of claim 27, wherein the active ingredient of the first component is a prodrug of mycophenolate mofetil, having the formula 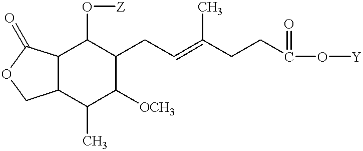
wherein Y is 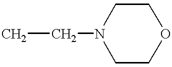
and Z is hydrogen or —(CO)R and R is lower alkyl or aryl.

29. The kit of claim 19, wherein the active ingredient of the first component is a compound of the formula 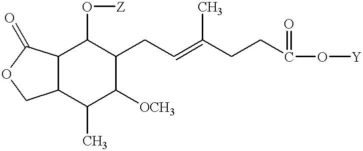
wherein Y is 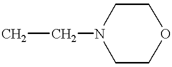
and Z is hydrogen.

30. The kit of claim 27, wherein the active ingredient of each injectable solution dose is a pegylated interferon-α2a.

31. The kit of claim 30, wherein the active ingredient of each injectable solution dose is a pegylated interferon-α2a conjugate of the formula 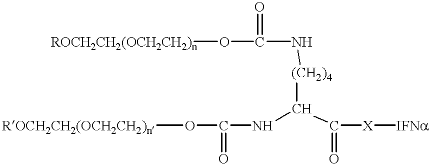
wherein R and R′ are independently lower alkyl; X is NH or 0; n and n′ are integers having a sum of from 600 to 1500; and the average molecular weight of the polyethylene glycol in said conjugate is from about 26,000 daltons to about 66,000 daltons.

32. The kit of claim 31, wherein the active ingredient of each injectable solution dose is a pegylated interferon-α2a conjugate of the formula 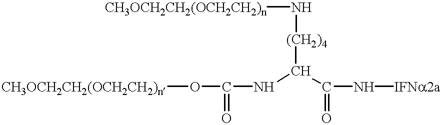
wherein n and n′ are independently 420 or 520.

33. A kit comprising:
a first component containing one or more oral unit dosage forms of an active ingredient, each unit containing about 250 mg to about 2000 mg of the active ingredient, wherein the active ingredient is a compound of the formula 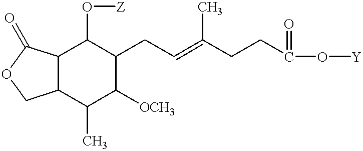
wherein Y 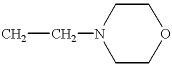
and Z is hydrogen, and
a second component containing a vial or series of vials each vial containing a single injectable solution dose or multiple injectable solution doses, each dose containing as an active ingredient about 40 μg to about 270 μg of a pegylated interferon-α conjugate of the formula 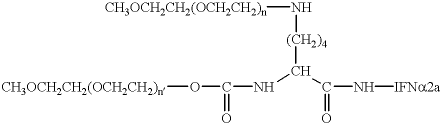
wherein n and n′ are independently 420 or 520.

34. The kit of claim 33, wherein the first component contains a sufficient a sufficient number of units so that a patient can administer about 2 grams per day of the compound for a period of about one to about four weeks and the second component contains a sufficient number of doses so that a patient can administer about 180 μg per week of the pegylated interferon-α conjugate for a period of about one to about four weeks.
